(19) United States
(12) Patent Application Publication
(10) Pub. No.: US 2026/0325645 A1
(43) Pub. Date: Oct. 22, 2026

(54) IMPROVEMENTS IN OR RELATING TO COMPOSITIONS FOR IMPROVEMENT OF PHOTODYNAMIC THERAPY

(71) Applicants: Anton ALEXANDROFF, Cambridge (GB); SYDERMA LTD, London (GB)

(72) Inventor: Anton ALEXANDROFF, Cambridge (GB)

(21) Appl. No.: 19/486,813

(22) PCT Filed: May 29, 2024
(86) PCT No.: PCT/GB2024/051371

§ 371 Date: Nov. 21, 2025

(30) Foreign Application Priority Data
Jun. 3, 2023 (GB) 2308327.2

(51) Int. Cl. A61K 41/00; A61K 31/522; A61P 17/00; A61P 17/10

(52) U.S. Cl. CPC A61K 41/0061; A61K 31/522; A61P 17/00; A61P 17/10

(57) ABSTRACT

Disclosed is a composition comprising photodynamic therapy photosensitizer and a composition comprising theobromine for treatment of a disorder of a mammalian, preferably human, subject; a composition comprising both photodynamic therapy photosensitizer and theobromine, for treatment of disorder of a mammalian, preferably human, subject; a method of making said composition; and a method of treating a disorder of a mammalian, preferably human, subject, using the composition/s of the invention.

### FIELD OF THE INVENTION

The present invention relates to a composition for treatment of lesions and conditions, to a method of making the composition, and to uses of the composition to improve photodynamic therapy.

### BACKGROUND OF THE INVENTION

Photodynamic therapy (PDT) is a technique based on the sequential application or administration of a photosensitizing drug or a precursor followed by exposure to a light. In dermatology it has been used to treat actinic keratosis disorders, basal cell carcinomas tumours, immune skin disorders such as acne, rosacea, folliculitis decalvans and psoriasis, immune mucosal membrane disorders such as oral lichen planus, genital lichen sclerosus, autoimmune skin disorders such as vitiligo and lupus erythematosus, as well as denture stomatitis, skin infections such as viral warts, fungal infection of skin, fungal keratitis, candida infections and Leishmania skin infection, various eye disorders and tumours including choroidal hemangiomas, retinal capillary hemangiomas, age related macular degeneration, and for the correction of cosmetic changes of aging and photoaging such as wrinkles (1).

However, PDT efficacy is limited. For instance, PDT clears 74% of actinic keratosis, gives less than 50 percent improvement with multiple treatments in vitiligo, 74-88% with multiple treatments in acne, (2,3,4). Therefore, there is a need to improve the efficacy of PDT treatment.

Photodynamic therapy relies on the basic principles of induction of reactive oxygen species (ROS) in cells that in turn damages the target cells. The excitation of the photosensitiser molecule results in a series of changes that creates OH▾ and O₂▾ radicals which in turn oxidise the macromolecules in the vicinity (5). Damaged proteins and RNA can be turned over and degraded by cells, therefore cells as such have a significant tolerance to such damage (6). DNA however cannot be turned over but needs to be repaired in situ, so such damage is the limiting factor in the cell survival and resistance to the treatment.

Oxidative damage of the DNA is a common lesion, occurring up to 100,000 times per day in a single cell (6). To this end, cells have evolved multiple mechanisms to resolve such events. The mechanisms of lesion induction through ROS are various and can result in more than 70 different adducts but, depending on the resulting chemical change to the target moiety and how it affects DNA structure, the lesion can either can be repaired through Base Excision Repair (BER) or Nucleotide Excision Repair (NER). BER predominantly repairs lesion that do not affect overall DNA structure while NER repairs alterations that distort the standard DNA alpha helix and create strain on neighbouring base pairings (6).

Although separate mechanisms with most of the components not shared between them, BER and NER share two common features in their repair process. The first one they induce single strand DNA breaks during DNA resection and repair (6). These are transient in nature, however during the acute ROS damage event, these can occur in clusters along the DNA. Such clusters can then result in more prolonged sections of single strand DNA in the case of multiple resection events on the same DNA strand or may result in bona fide double strand DNA breaks (DSBs) if they occur on opposing strands in close proximity (6 and references therein). This fits well with the results showing that during acute ROS insults both ATM and ATR kinases can be activated in different cell lines (7) although the level of cellular response and outcomes are highly variable and not predictable beforehand. Among others, ATR kinase can be activated by extended stretches of single strand DNA (ssDNA), while ATM had been shown to be a key master regulator of DSB repair and is activated at DSB sites (8). Although ROS damage induces base changes that can be removed through BER and NER, data showing that impairment of either ATM or ATR can in some cell lines lead to higher sensitivity to the ROS damage (6) indicates that indeed ROS damage can lead to more complex DNA damage structures that require either of the kinases for their resolution.

The second feature is that both are affected by the activity of Poly-ADP ribose polymerase (PARP). PARP is a regulator of multiple repair pathways, affecting not just BER and NER, but repair pathways as well, including DSB repair mechanisms (18 and references therein). Its pleiotropic activity extends beyond repair mechanisms, where it also mediates necrotic cell death (19). Notably, inhibition of PARP affects multiple repair pathways, including BER (20), NER (21), NHEJ (22) and HR (23), reducing their efficacy.

Activation of either ATR or ATM in the context of DNA damage has multiple key cellular responses. One of those is immediate cell cycle arrest via the corresponding ATR-CHK1 or ATM-CHK2 signalling axes (7). Repair of DSB can be rapid, but can also be extended to the period of hours, depending on the complexity of the lesion. Therefore, cell cycle arrest serves to stabilise the sites of damage while they are in the process of repair and prevent them from entering either DNA replication or cell division, either of which could be detrimental if they occur in the presence of unresolved DNA lesions (8). Depending on the extent of damage, the capacity of the repair mechanisms in any given cells and other factors, this may or may not lead to activation of programmed cell death or apoptosis in the damaged cell. Critically, the inhibition of the kinases have in certain instances resulted in higher cellular sensitivity to ROS (6).

Like other DNA damaging agents, sensitivity to PDT correlates with the ability of targeted cells to repair incurred damage. This can depend on the activity of underlying repair mechanisms, but may also depend indirectly on their proliferative state. Cells that do not divide tend to tolerate DNA damage much more than cells that actively proliferate, who by virtue of pressure to replicate DNA and divide have a shorter time window to either repair the damage or, alternatively, undergo cell death through various mechanisms if they can't. In case of hyperplastic disorders, the treatment target is hyperproliferating cells that are the direct cause of the disease. In immune disorders, the target are immune cells, which by virtue of hyperactivation are stimulated to significantly proliferate (9 and references therein). In both cases it is expected that the repair of oxidative damage would at least in part go via the ATM and ATR kinases, the key regulators of repair of complex DNA lesions, including DSBs (8).

Multiple approaches to treatment of hyperproliferative disorders using DNA repair inhibitors are known in the art, however they all unequivocally focus on specific inhibitors of individual pathways. However, in case of PDT treatment, the inventors postulated that using the broad-spectrum inhibitor, that would comprise in one compound an inhibitor of ATM, ATR and PARP enzymatic activities, would work best to enhance PDT activity. To this end, only one compound was identified to have this capability, which is theobromine.

Theoobromine is a member of methylxantine family of compounds, which are methylated derivatives of xanthine base and can occur naturally, like caffeine, theobromine and theophylline, or can be synthetic, like pentoxyphilline. Both caffeine and theobromine have been shown the be inhibitors of both ATM and ATR (10), however theobromine is the only compound with measurable PARP inhibitory activity in addition to both ATM and ATR inhibitory activity. Both aforementioned compounds, together with theophylline and pentoxyphilline have all been shown to be radiosensitisers (11,12,13 and references therein). Pentoxyfiline has also been tested as radiosensitiser in clinical trials but has failed to provide consistent benefits (17 and references therein). The effects of other methylxanthine compounds on the full spectrum DNA repair mechanisms is not known as of yet.

### SUMMARY OF THE INVENTION

To increase effectiveness of photodynamic therapy the inventors set out to reduce the ability of cells to repair photodynamic therapy-mediated oxidative stress and resultant DNA damage, including DSBs. The inventors hypothesised that one way to achieve this might be to prevent cellular recognition of these lesions via activation of Ataxia Telangiectasia Mutated (ATM) and Ataxia Telangiectasia Related (ATR) kinases. A class of such inhibitors are methylxanthines, in particular theobromine, which have been shown to inhibit DNA repair in vitro, through modulating the activity of ATM/ATR kinases, as well as PARP (10,11,12,14).

Accordingly, in a first aspect, the invention provides a first composition comprising a photodynamic therapy photosensitizer preferably methyl aminolevulinate or 5-aminolaevulinic acid or other photosensitizers applied as a topical application and a second composition comprising the triple (ATM, ATR, PARP) inhibitor, for treatment of skin and/or mucous membranes disorders and/or lesions and disorders elsewhere in the body. A preferred photodynamic therapy photosensitizer is methyl aminolevulinate or 5-aminolaevulinic acid but other photosensitizers including but not limited to hematoporphyrin derivative, m-tetrahydroxophenyl chlorine, mono-L-aspartyl chlorine e6, fotosens, phthalocyanines, benzoporphyrins, chlorins, bacteriochlorins, naphthalocyanines, furanocoumarins, verteporfin can also be used. A preferred triple (ATM, ATR, PARP) inhibitor is a methylxanthine, more especially theobromine. In some embodiments, the second composition may comprise two or more inhibitors, one or more of which may be a methylxanthine, preferably theobromine. In some embodiments, the first composition may comprise two or more photodynamic therapy photosensitizers including methyl aminolevulinate, 5-aminolaevulinic acid, hematoporphyrin derivative, m-tetrahydroxophenyl chlorine, mono-L-aspartyl chlorine e6, fotosens, phthalocyanines, furanocoumarins or other photosensitizer; most preferably methyl aminolevulinate.

The compositions may be applied topically to the skin and/or mucous membrane of a mammalian subject. The subject will advantageously be a human, usually an adult human, or a teenager. Mammalian subjects other than humans may be, for instance, companion animals or farming livestock. Examples of treated disorders of skin and/or mucous membranes, which may be treated using the compositions of the invention include, but are not limited to, the following: actinic keratosis; basal cell carcinoma; acne.

The compositions will preferably be formulated for topical application, using formulation methodologies and non-active ingredients which are conventional in the art. Thus, for example, the compositions may be formulated as a gel, cream, ointment, lotion, solution, suspension, plasters, nanoparticles, microneedles or the like and, in addition to the active agents, may otherwise typically comprise conventional constituents such as water, emulsifiers (e.g. polysorbate compounds, especially polysorbate 20-polysorbate 80), preservatives and antimicrobials (e.g. parabens, such as methyl hydroxybenzoate, propyl hydroxybenzoate), a dermatologically acceptable carrier, diluent or excipient, (e.g. propylene glycol, lanolin), and thickeners and emollients (e.g. stearyl alcohol). Appropriate amounts and relative proportions of these substances will be apparent to those skilled in the art and/or can be readily determined by trial and error.

The compositions may be applied separately to the affected area of the body. The sequence of application is not important. Alternatively, both compositions may be applied or administered essentially simultaneously (i.e. the second composition is applied within 10 minutes of applying the first composition). Advantageously the compositions will be both applied to the affect part of the skin within 24 hours of one another. The compositions will be applied within 24 hours of light exposure, or more preferably within 30 min to 2 hours before light exposure. Daylight or various photodynamic therapy light sources can be used. Typically, the two respective compositions will be applied to the affected area of the body on the same day, once, or if advantageous once weekly or every other week, it can also be applied monthly or every other month or every 3 months until a course of treatment is completed; the triple (ATM, ATR, PARP) inhibitor is applied every day or every other day until a course of treatment is completed. In some instances the triple (ATM, ATR, PARP) inhibitor can be applied on same day as a photodynamic therapy photosensitizer and also daily for 1 or 3 or 5 or 7 days after the application of the photosensitizer until a course of treatment is completed. Light irradiation is typically administered within 24 hours of photodynamic therapy photosensitizer and more preferably within 30 minutes to 2 hours after photodynamic therapy photosensitizer administration.

In a second aspect, the invention provides a single composition comprising both photodynamic therapy photosensitizer and the triple (ATM, ATR, PARP) inhibitor. Preferably the triple (ATM, ATR, PARP) inhibitor is a methylxanthine, more especially theobromine. In some embodiments, the composition may comprise two or more inhibitors, one or more of which may be the triple (ATM, ATR, PARP) methylxanthine inhibitor, preferably theobromine. In some embodiments, the composition may comprise two or more photodynamic therapy photosensitizer including but not limited methyl aminolevulinate, 5-aminolaevulinic acid hematoporphyrin derivative, m-tetrahydroxophenyl chlorine, mono-L-aspartyl chlorine e6, fotosens, furanocoumarins, verteporfin, methylene blue; most preferably methyl aminolevulinate.

The composition of the second aspect of the invention may preferably be for treatment of skin and/or mucous membranes disorders in a mammalian, preferably human, subject. The features of the first aspect of the invention defined above will, unless the context dictates otherwise, be equally applicable to the composition of the second aspect.

The composition/s of the first or second aspects of the invention may comprise photodynamic therapy photosensitizer and/or the triple (ATM, ATR, PARP) inhibitor as the sole active agent, or may additionally comprise one or more other substances known to have activity against hyperplastic keratinocyte disorders, autoimmune disorders, such as actinic keratosis, acne or, basal cell carcinomas or other tumours when topically applied. For example, other substances known to have activity against actinic keratosis include: diclofenac, imiquimod (e.g. at 3-5% v/w), salicylic acid (e.g. at up to 10% v/w), ingenol mebutate (e.g. at 0.015-0.05 % v/w), and tirbanibulin (e.g. at 1% v/w). In another example, other substances known to have activity against acne include benzoyl peroxide, adapalene and other topical retinoids, topical or oral antibiotics including clindamycin, erythromycin, lymecycline, oral isotretinoin, dapsone and other treatments.

The composition/s will comprise a photodynamic therapy photosensitizer and/or the triple (ATM, ATR, PARP) inhibitor, at an effective concentration, that is, a concentration which, when the composition is applied to the patient, brings about a measurable improvement in the condition after a suitable period of treatment (typically this will involve daily topical application, for at least 10 minutes, preferably 30 minutes and more preferably 2 hours. In other instances, it can be more advantageous to apply the composition/s for least 1 day, a few days, 1 week, 2 weeks, and in some instances at least three weeks). In some instances it would be preferable to repeat application of the composition/s after a suitable period of time like 1 day or preferably 1 week and more preferably 1 month and most preferably 3 months. The composition/s will comprise a photodynamic therapy photosensitizer and/or the triple (ATM, ATR, PARP) inhibitor, at an effective concentration, and will be applied or administered to the patient at a suitable period of time before light irradiation (typically it would involve applying or/and administering the compositions between 1 minute and 24 hours before light irradiation, and more preferably 10 to 360 minutes before light irradiation and most preferably 30 minutes and 2 hours before light irradiation). Improvement in the condition being treated can be determined, for example, by measuring the area of the visible lesion or lesions, or by counting the number of visible lesions in the treated area of skin or body, or by measuring a decreasing volume of tumour.

Typically an effective concentration of a photodynamic therapy photosensitizer will be in the range 0.01 to 50% w/w or w/v, preferably in the range 0.1 to 30%, and more preferably in the range 1 to 20%, and most preferably around 7-16%.

An effective concentration of the triple (ATM, ATR, PARP) inhibitor will typically be in the range 0.001 to 50% w/w or w/v, preferably in the range 0.01 to 10%, and more preferably in the range 0.05 to 1%, and most preferably around 0.1-0.5%.

In a third aspect, the invention provides a method of making a composition in accordance with the second aspect of the invention, the method comprising the step of: causing to be present, in a single composition, an effective concentration of a photodynamic therapy photosensitizer and an effective concentration of the triple (ATM, ATR, PARP) inhibitor.

Generally, the method of making a composition as defined above will further comprise mixing a photodynamic therapy photosensitizer and the triple (ATM, ATR, PARP) inhibitor with a dermatologically acceptable carrier, diluent or excipient, and packaging the composition in a container, such as a tube, bottle, tub or the like. If necessary such compositions/s may have to be sterilised by filtering them through an antibacterial filter or by other suitable means.

In a fourth aspect, the invention provides a method of treating hyperproliferative disorders of keratinocytes in skin and/or mucous membranes and/or immune disorders including but not limited to actinic keratoses, acne, basal cell carcinoma, of a mammalian, preferably human, subject, the method comprising the step of applying to the skin and/or mucous membrane or as a systemic administration, as appropriate, of a patient in need of such treatment, an effective amount of first and second compositions in accordance with the first aspect of the invention, or an effective amount of a single composition in accordance with the second aspect of the invention, as defined above, and irradiating the appropriate area/s of skin and/or mucous membranes by a suitable source of light including visible light sources or daylight for a suitable duration of time.

Some agents contained within the composition have been approved or have had history of safe use for topical application so the treatment may be applied by the patient themselves. Alternatively, they may be applied by a medical professional or veterinarian. Typically, the composition is applied to the affected area of skin and/or mucous membrane once for a period of at least few minutes or hours or up to 1 day, but in some instances it can be administered on a daily basis, or possibly every other day, or other time intervals, up to one week or up to 3 months. Alternatively, the treatment may be repeated weekly or monthly or every 3 months for a desired period of time. Following a topical application of the composition/s the skin or mucous membrane condition is irradiated by a photodynamic suitable light source which could be a specialised photodynamic light source or a daylight. Typically, the treated conditions or tumours are exposed to a suitable for photodynamic therapy light source after a suitable time period typically between 5 minutes and 24 hours, and more preferably 20 minutes and 4 hours and most preferably 30 minutes and 2 hours after applying or administering the composition/s. At the end of this period the patient may be assessed by a medical professional (or veterinarian, as appropriate) if desired, and treatment either continued or halted, as appropriate, according to the opinion of the medical professional or veterinarian. In some instances the patient may be assessed a week or a month after completing treatment, or at other time intervals.

Advantages of the various aspects of the present invention include: more effective treatment than existing therapies.

In particular, the inventors have found that the combination of a photodynamic therapy photosensitizer and the triple (ATM, ATR, PARP) inhibitor confers synergistic results, greater than the additive effects of the two treatments in isolation. In particular the triple (ATM, ATR, PARP) inhibitor on its own has not improved actinic keratoses or acne.

The invention will now be further described by way of illustrative examples and with reference to the accompanying drawings in which:

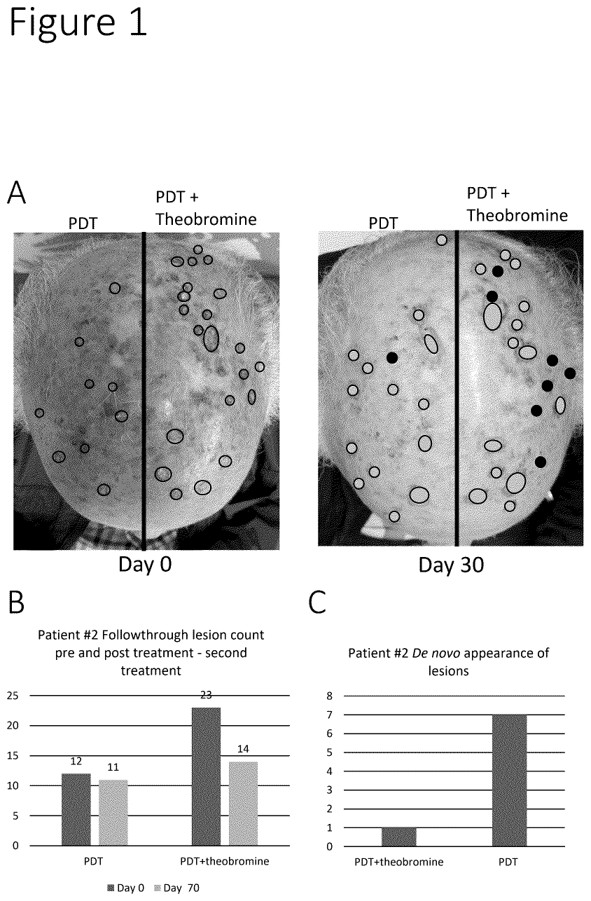

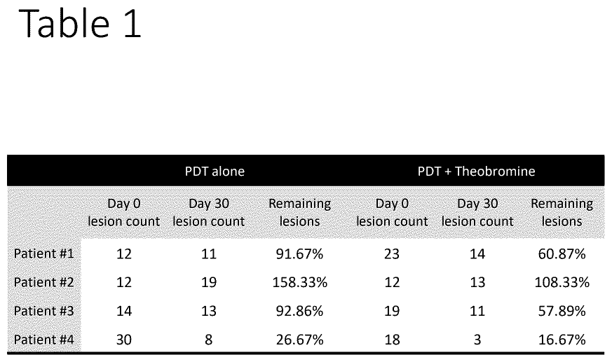

FIG. 1: FIG. 1A shows images of actinic keratosis lesions treated with either methyl aminolevulinate alone, or a combination of methyl aminolevulinate and theobromine after a period of time as indicated. Light-filled circles represent positions of individual identified actinic keratosis lesions and dark-filled circles represent lesions that have been identified as removed upon treatment on either side of the target region. FIG. 1B shows quantitation of lesions identified at Day 0 followed through to Day 30. FIG. 1C show the number of de novo lesions appearing in the same region after treatment, as described in Example 1 below;

Table 1: Observational data from Patients #1-4, as described in Examples 1 and 2 below;

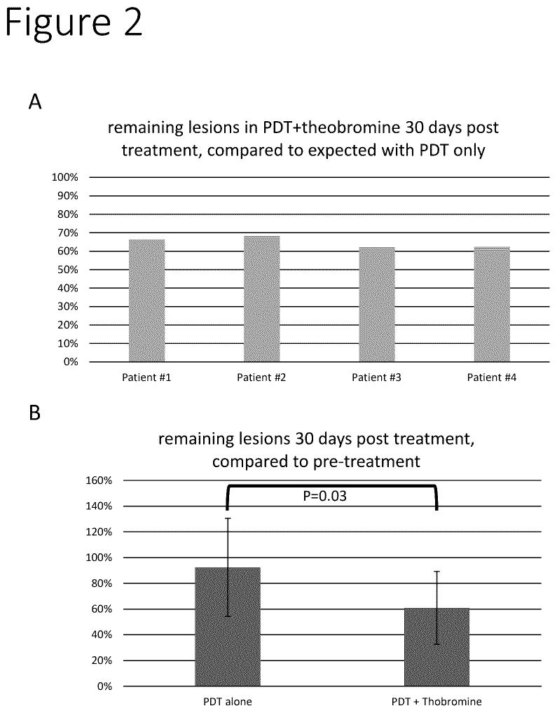

FIG. 2: FIG. 2A shows the fraction observed levels of remaining AK lesions upon the combination treatment of aminolevulinate (Photodynamic therapy—PDT) and theobromine, as compared to expected levels when compared to the control region in each corresponding patient. FIG. 2B shows the combined results from patients #1-4. Error bars represent standard deviation and the statistical relevance, as measured by a paired T-test is 0.03. Results are as described in Example 2 below;

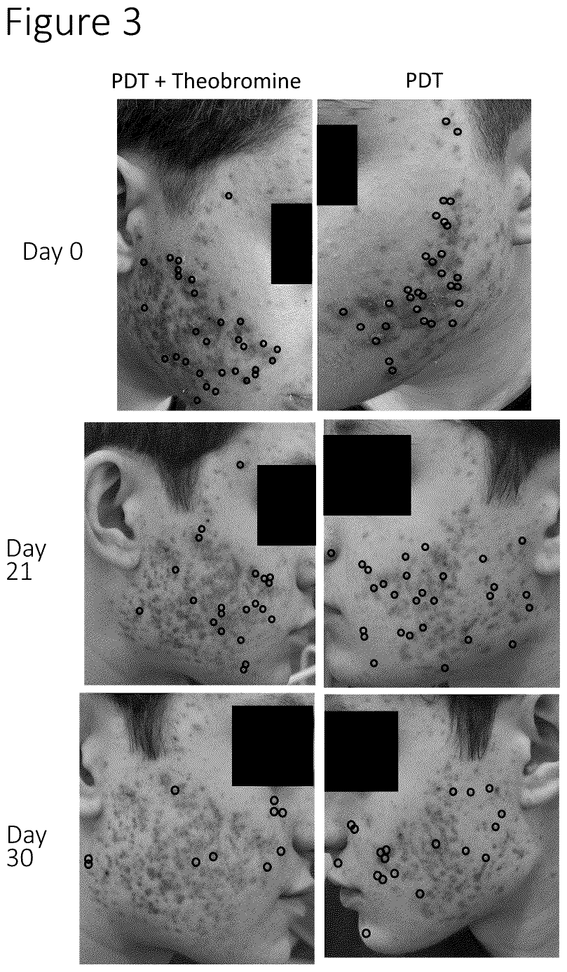

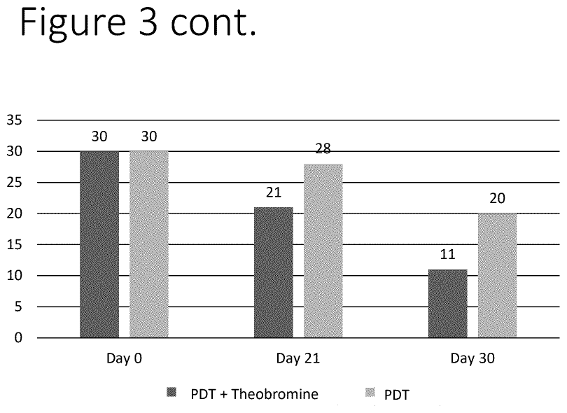

FIG. 3: FIG. 3A shows images of acne lesions treated with either methyl aminolevulinate (PDT) alone, or a combination of methyl aminolevulinate and theobromine after periods of time as indicated. Dark circles represent positions of individual active acne lesions. FIG. 3B shows quantitation of lesions at Day 0, followed through to Day 30, as described in Example 3 below;

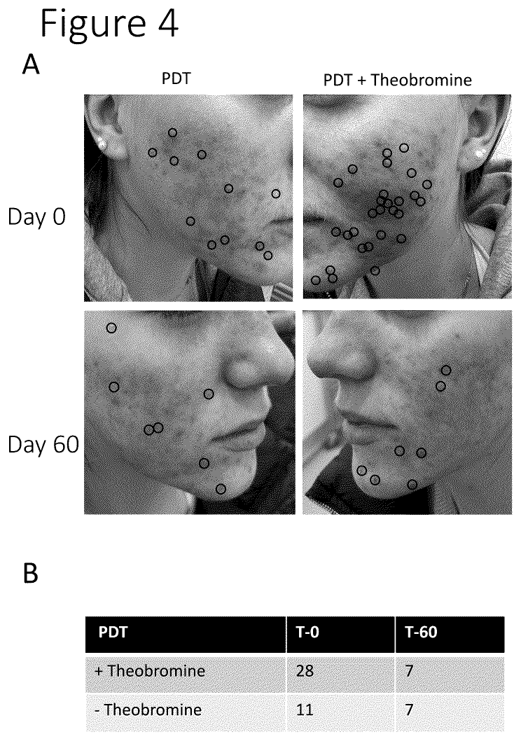

FIG. 4: FIG. 4A shows images of acne lesions treated with either methyl aminolevulinate (PDT) alone, or a combination of methyl aminolevulinate and theobromine after periods of time as indicated. Circles represent positions of individual active acne lesions. FIG. 4B shows quantitation of lesions at Day 0 and Day 60 after treatment, as described in Example 6 below;

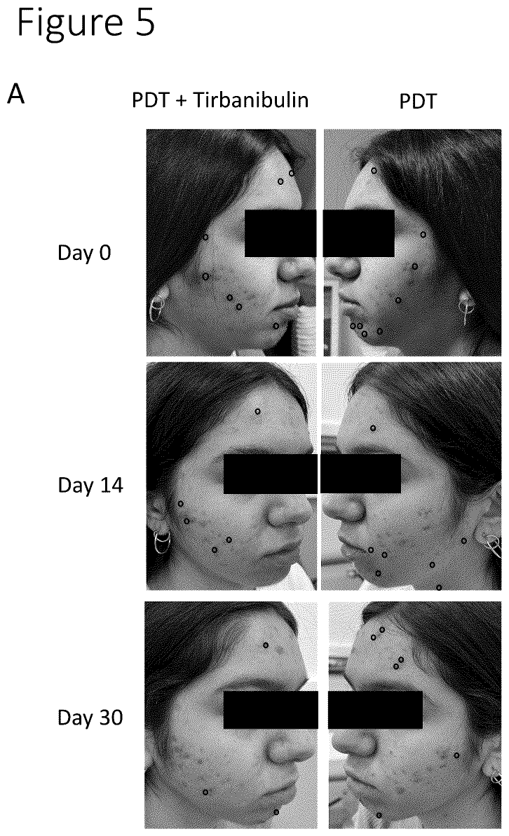

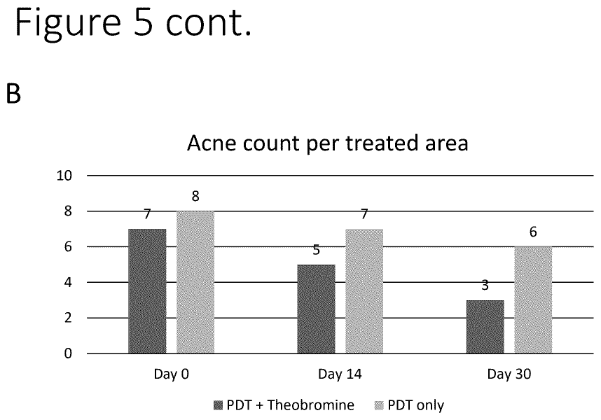

FIG. 5: FIG. 5A shows images of acne lesions treated with either methyl aminolevulinate (PDT) alone, or a combination of methyl aminolevulinate (PDT) and theobromine after periods of time as indicated. Circles represent positions of individual active acne lesions. FIG. 5B shows quantitation of lesions at Day 0, Day 14 and Day 30 after treatment, as described in Example 7 below;

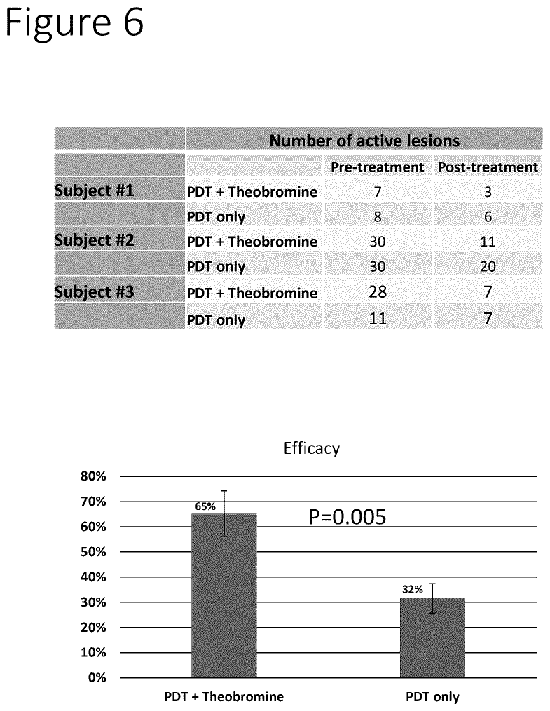

FIG. 6: FIG. 6A shows pre-and final post-treatment data as presented in Examples 3, 6 and 7. FIG. 6B show data analysis of the data presented in FIG. 6A. Data show the combination of methyl aminolevulinate (PDT) and theobromine result in clearance of on average 65% of active lesions, while methyl aminolevulinate alone clears only 32%. The P-value as calculated by the paired, two-tailed T-test is 0.005, showing significant enhancement if efficacy of the combined treatment compared to the PDT only standard-of-care control.

### EXAMPLE 1

A 76-year old male patient with presented with punctate simple actinic keratosis (AK) lesions bilaterally across the scalp the lesions, identifiable by position but not quantifiable by size due to potential inaccuracies in edge identification. For the purposes of the test, the affected area was split into two portions, left and right scalp. Both parts of the area were treated with the photosensitizer 16% w/w methyl aminolevulinate hydrochloride cream (Metvix®), and the left segment also in combination with 0.5% theobromine topical preparation solution. The theobromine solution was prepared in 25% ethanol and 50% glycerol, adjusted to pH 8 via 100 mM sodium hydrogen carbonate solution.

For treatment, theobromine solution was applied to the left side of the scalp in a thin aqueous film using 200-300 μL of the solution (4-6 drops). The solution was left to dry and permeate the skin for 20 minutes, after which both sides of the forehead were covered in the photosensitizer cream as per manufacturer's instructions, using 1-1.5 g of cream. The cream was left to permeate the skin for 5-10 minutes and the patient was asked to spend 2 h outside in the natural sunlight to induce optimal activation of the photosensitizer in the skin. The patient was advised to stay indoors afterwards and wash the face to remove any remaining active ingredients.
The patient was assessed first prior to treatment and subsequently 30 days after for the success and quantification of AK lesions on each side of the scalp by a qualified dermatologist. Initial assessment showed 12 lesions on the right side of the scalp and 23 lesions on the left side of the scalp. The analysis 30 days after treatment showed 11 remaining lesions of the 12 treated on the right side, while 14 lesions were remaining out of the original 23 on the left. Notably, there was no comparative difference in erythema or pain/irritation over the 30 days post treatment and the patient did not report any adverse reactions on either arm of the experiment.

Patient also presented with de novo AK flare up, however 8 AK lesions were observed on the side treated with PDT while only 1 lesion was seen on the side treated with theobromine. This indicates that the combination not only is capable of removing AK lesions to a greater degree, but also is capable of preventing de novo lesions occurring, reducing patient risk further.

### EXAMPLE 2

Three further patients (Patients #2, #3 and #4) ages 56, 78 and 70, presented with multiple AKs across the scalp and were treated as in Example 1. All three patients presented with multiple simple AK lesions, which were not identifiable by size, so were solely assessed for their presence before and after treatment. As prior, with Patients #2 and #3 treatment was separated medially, however in their case the right side treated with 0.5% theobromine 20-30 minutes before treatment with the photosensitizer 7.8% w/w 5-aminolevulonic acid hydrochloride gel (Ameluz®) bilaterally across the scalp. The Patient #4 was treated using a similar protocol, but with 0.5% theobromine applied posterior on the scalp prior to photosensitizer treatment. All other parts of the treatment were identical to Example 1, including compositions of and formulations of theobromine and application protocols. AK lesions were quantified in all patients at the time of treatment as 30 days post treatment for effectiveness. The results are as presented in Table 1.

Upon analysis, the efficacy of photosensitiser treatment was quite varied from patient to patient, varying from no effectiveness to 73% effectiveness (Table 1). As the photodynamic treatment was performed at normal daylight, self-regulated by patients under the instructions of a medical professional and affected with day-to-day variations in weather conditions and light intensity, the inventors ascribe the apparent variation to the combination of the above variables. This notwithstanding, the combination treatment showed and surprisingly constant improvement over PDT alone, exhibiting the level of unresolved AKs at the level of 65-70% of the expected amounts, across a wide range of PDT only efficacy ((FIG. 2A). When analysed by a paired T-test, the results showed a significant improvement (p=0.03) in the PDT+Theobromine treatment versus PDT alone, indicating a quantifiable, unexpected positive impact of localised topical theobromine pre-treatment on PDT treatment efficacy.

### EXAMPLE 3

A 18-year old male patient presented with acne bilaterally on his face. The acne presented as active inflammatory reaction or as visible white liquid-filled extrusions. For the purposes of the test, the affected area was split into two sections, left cheek and right cheek. Both areas were treated with the photosensitizer 16% w/w methyl aminolevulinate hydrochloride cream (Metvix®), and the right face also with combination with 0.5% theobromine topical preparation solution. The theobromine solution was prepared in 25% ethanol and 50% glycerol, adjusted to pH 8 via 100 mM sodium hydrogen carbonate solution.

For the purposes of the treatment, theobromine solution was applied to the right side of the face in a thin film using 100-200 μL of the solution (2-4 drops). The solution was left to dry and penetrate the skin for 20 minutes, after which both sides of the face were covered in the photosensitizer cream as per manufacturer's instructions, using 1-1.5 g of cream. The cream was left to penetrate the skin for 5-10 minutes and the patient was asked to spend 2 h outside in the natural sunlight to induce optimal activation of the photosensitizer in the skin. The patient was advised to stay indoors afterwards and wash the face to remove any remaining active ingredients.

The patient was assessed first prior to treatment and subsequently 21 days and 30 days after for the success and quantification of active acne on each side of the face by a qualified dermatologist. Initial assessment showed 30 active acne lesions on the right side of the face and 30 lesions on the left side. The analysis 21 days after treatment showed 28 active sites on the right side, while 21 active sites on the left. Further analysis at day 30 showed 20 active lesions on the right side and 11 on the left. No patient-reported adverse effects were identified and clinically only mild erythema at day 7 post treatment was detected, which subsided by day 14. Furthermore, active inflammation areas of the skin at both days 21 and 30 were less prominent on the right side of the face, as assessed by a medical professional (FIG. 3)

### EXAMPLE 4: (COMPARATIVE EXAMPLE, NOT IN ACCORDANCE WITH THE INVENTION)

A 62 year old man presented with actinic keratoses on his scalp. Three actinic keratoses were selected by a qualified dermatologist and treated once with 0.5% theobromine topical preparation solution. The theobromine solution was prepared in 25% ethanol and 50% glycerol, adjusted to pH 8 via 100 mM sodium hydrogen carbonate solution. For treatment, theobromine solution was applied to the selected lesions of the scalp in a thin aqueous film using 50 μL of the solution per lesion (1 drop). The solution was left to dry and permeate the skin for 20 minutes. The position of the lesions was marked with a water-soluble marker and the lesions were photographed. The patient was reviewed 1 month later by a qualified dermatologist and the lesions were compared with the baseline photographs. No redness or soreness were observed. No improvement of actinic keratoses was observed.

### EXAMPLE 5: (COMPARATIVE EXAMPLE, NOT IN ACCORDANCE WITH THE INVENTION)

A 61 year old man presented with actinic keratoses on his face and scalp. An actinic keratosis was selected by a qualified dermatologist on his right cheek and treated once with 0.5% theobromine topical preparation solution. The theobromine solution was prepared in 25% ethanol and 50% glycerol, adjusted to pH 8 via 100 mM sodium hydrogen carbonate solution. For treatment, theobromine solution was applied to the selected lesions of the scalp in a thin aqueous film using 50 μL of the solution per lesion (1 drop). The solution was left to dry and permeate the skin for 20 minutes. The position of the lesion was marked with a water-soluble marker and the lesion was photographed. The patient was reviewed 1 month later by a qualified dermatologist and the lesion was compared with the baseline photographs. No redness or soreness were observed. No improvement of actinic keratosis was observed.

### EXAMPLE 6

A 18-year old female patient presented with acne bilaterally on her face. The acne presented as active inflammatory reaction or as visible white liquid-filled extrusions. For the purposes of the test, the affected area was split into two sections, left cheek and right cheek. Both areas were treated with the photosensitizer 7.8% w/w 5-aminolevulonic acid hydrochloride gel (Ameluz®), and the left face also with combination with 0.5% theobromine topical preparation solution. The theobromine solution was prepared in 25% ethanol and 50% glycerol, adjusted to pH 8 via 100 mM sodium hydrogen carbonate solution.

For the purposes of the treatment, theobromine solution was applied to the left side of the face in a thin film using 100-200 μL of the solution (2-4 drops). The solution was left to dry and penetrate the skin for 20 minutes, after which both sides of the face were covered in the photosensitizer cream as per manufacturer's instructions, using 1-1.5 g of cream. The cream was left to penetrate the skin for 5-10 minutes and the patient was asked to spend 2 h outside in the natural sunlight to induce optimal activation of the photosensitizer in the skin. The patient was advised to stay indoors afterwards and wash the face to remove any remaining active ingredients.

The patient was assessed first prior to treatment and subsequently 2 months after for the success and quantification of active acne on each side of the face by a qualified dermatologist. Initial assessment showed 11 active acne lesions on the right side of the face and 28 lesions on the left side. The analysis 2 months after treatment showed 7 active sites on the right side, while 7 active sites on the left. No patient-reported adverse effects were identified. The patient also self evaluated severity of acne on the scale of 10. The patient evaluated severity of acne at the baseline as 7/10 on the right and 9/10 on the left. Two months after treatment the patient evaluated severity of acne as 5/10 on the right and 3/10 on the left.

### EXAMPLE 7

A 19-year old female patient presented with acne bilaterally on his face. The acne presented as active inflammatory reaction or as visible white liquid-filled extrusions. For the purposes of the test, the affected area was split into two sections, left cheek and right cheek. Both areas were treated with the photosensitizer 7.8% w/w 5-aminolevulonic acid hydrochloride gel (Ameluz®), and the right face also with combination with 0.5% theobromine topical preparation solution. The theobromine solution was prepared in 25% ethanol and 50% glycerol, adjusted to pH 8 via 100 mM sodium hydrogen carbonate solution.

For the purposes of the treatment, theobromine solution was applied to the right side of the face in a thin film using 100-200 μL of the solution (2-4 drops). The solution was left to dry and penetrate the skin for 20 minutes, after which both sides of the face were covered in the photosensitizer cream as per manufacturer's instructions, using 1-1.5 g of cream. The cream was left to penetrate the skin for 5-10 minutes and the patient was asked to spend 2 h outside in the natural sunlight to induce optimal activation of the photosensitizer in the skin. The patient was advised to stay indoors afterwards and wash the face to remove any remaining active ingredients.

The patient was assessed first prior to treatment and subsequently 14 days and 30 days after for the success and quantification of active acne on each side of the face by a qualified dermatologist. Initial assessment showed 7 active acne lesions on the right side of the face and 8 lesions on the left side. The analysis 14 days after treatment showed 5 active sites on the right side, while 7 active sites on the left. Further analysis at day 30 showed 3 active lesions on the right side and 6 on the left. No patient-reported adverse effects were identified. Furthermore, active inflammation areas of the skin at both days 14 and 30 were less prominent on the right side of the face, as assessed by a medical professional.

### EXAMPLE 8 (COMPARATIVE EXAMPLE)

A 17-year old female patient presented with acne bilaterally on her face. The acne presented as active inflammatory reaction or as visible white liquid-filled extrusions. For the purposes of the test, the affected area was split into two sections, left cheek and right cheek. The right face was treated with 0.5% theobromine topical preparation solution. The theobromine solution was prepared in 25% ethanol and 50% glycerol, adjusted to pH 8 via 100 mM sodium hydrogen carbonate solution.

For the purposes of the treatment, theobromine solution was applied to the right side of the face in a thin film using 100-200 μL of the solution (2-4 drops). The solution was left to dry and penetrate the skin for 20 minutes, after which both sides of the face were covered in the photosensitizer cream as per manufacturer's instructions, using 1-1.5 g of cream. The cream was left to penetrate the skin for 5-10 minutes and the patient was asked not to wash her face for 2 hours.

The patient was assessed first prior to treatment and subsequently 1 month after for the success and quantification of active acne on each side of the face by a qualified dermatologist. Theobromine treatment did not improve acne (data not shown). No patient-reported adverse effects were identified.

### EXAMPLE 9 (COMPARATIVE EXAMPLE)

A 16-year old male patient presented with acne bilaterally on her face. The acne presented as active inflammatory reaction or as visible white liquid-filled extrusions. For the purposes of the test, the affected area was split into two sections, left cheek and right cheek. The left face was treated with 0.5% theobromine topical preparation solution. The theobromine solution was prepared in 25% ethanol and 50% glycerol, adjusted to pH 8 via 100 mM sodium hydrogen carbonate solution.

For the purposes of the treatment, theobromine solution was applied to the left side of the face in a thin film using 100-200 μL of the solution (2-4 drops). The solution was left to dry and penetrate the skin for 20 minutes, after which both sides of the face were covered in the photosensitizer cream as per manufacturer's instructions, using 1-1.5 g of cream. The cream was left to penetrate the skin for 5-10 minutes and the patient was asked not to wash her face for 2 hours.

The patient was assessed first prior to treatment and subsequently 1 month after for the success and quantification of active acne on each side of the face by a qualified dermatologist. Theobromine treatment did not improve acne (data not shown). No patient-reported adverse effects were identified.

### EXAMPLE 10 (COMPARATIVE EXAMPLE)

A 17-year old female patient presented with acne bilaterally on her face. The acne presented as active inflammatory reaction or as visible white liquid-filled extrusions. For the purposes of the test, the affected area was split into two sections, left cheek and right cheek. The right face was treated with 0.5% theobromine topical preparation solution. The theobromine solution was prepared in 25% ethanol and 50% glycerol, adjusted to pH 8 via 100 mM sodium hydrogen carbonate solution.

For the purposes of the treatment, theobromine solution was applied to the right side of the face in a thin film using 100-200 μL of the solution (2-4 drops). The solution was left to dry and penetrate the skin for 20 minutes, after which both sides of the face were covered in the photosensitizer cream as per manufacturer's instructions, using 1-1.5 g of cream. The cream was left to penetrate the skin for 5-10 minutes and the patient was asked not to wash her face for 2 hours.

The patient was assessed first prior to treatment and subsequently 1 month after for the success and quantification of active acne on each side of the face by a qualified dermatologist. Theobromine treatment did not improve acne (data not shown). No patient-reported adverse effects were identified.

### EXAMPLE 11 (COMPARATIVE EXAMPLE)

A 16-year old male patient presented with acne bilaterally on her face. The acne presented as active inflammatory reaction or as visible white liquid-filled extrusions. For the purposes of the test, the affected area was split into two sections, left cheek and right cheek. The right face was treated with 0.5% theobromine topical preparation solution. The theobromine solution was prepared in 25% ethanol and 50% glycerol, adjusted to pH 8 via 100 mM sodium hydrogen carbonate solution.

For the purposes of the treatment, theobromine solution was applied to the right side of the face in a thin film using 100-200 μL of the solution (2-4 drops). The solution was left to dry and penetrate the skin for 20 minutes, after which both sides of the face were covered in the photosensitizer cream as per manufacturer's instructions, using 1-1.5 g of cream. The cream was left to penetrate the skin for 5-10 minutes and the patient was asked not to wash her face for 2 hours.

The patient was assessed first prior to treatment and subsequently 6 weeks after for the success and quantification of active acne on each side of the face by a qualified dermatologist. Theobromine treatment did not improve acne (data not shown). No patient-reported adverse effects were identified.

### EXAMPLE 12

In a specific embodiment, a composition in accordance with the second aspect of the present invention comprises: (a) methyl aminolevulinate hydrochloride, present in the composition at a concentration of 16% w/w and (b) theobromine, present in the composition at a concentration of 0.5 % w/w, (a) and (b) being the active ingredients. In addition, the composition contains:
(c) olive oil (20% w/w);
(d) lanolin (6% w/w);
(e) glycerine 5% w/w);
(f) propylene glycol (20% w/w);
(g) polyethylene glycol 400 (23% w/w);
(h) isopropyl myristate (3% w/w); and
(i) an aqueous buffer solution adjusted to an appropriate pH (e.g. in the range 6.5-7.5).

Components of the formulation are filter sterilised by passage through a 0.22 mm pore filter as necessary and the formulation is stored at 4 °C until required.

The formulation of the example can be used to treat a subject suffering from hyperproliferative keratinocyte disorders of the skin and/or mucous membrane, such as actinic keratoses or acne.

The skin and/or mucous membrane, as appropriate, is treated with above topical preparation. The cream is topically applied to the entire area to be treated, at ambient temperature, about 18-20 °C. The formulation is left on the subject's skin for 20 minutes. and then the area is exposed to daylight for 2 hours. After this the desired the area or washed with soap and water and the patient remains indoors for the rest of the day. The treatment is applied once and the result of treatment is evaluated after 1 month.

### EXAMPLE 13

In a specific embodiment, a composition in accordance with the second aspect of the present invention comprises: (a) 5-aminolevulonic acid hydrochloride present in the composition at a concentration of 7.8% w/w; and (b) theobromine, present in the composition at a concentration of 0.5% w/w, with (a) and (b) being the active ingredients. In addition, the composition contains:
(c) isopropyl myristate (25% w/w);
(d) polyoxyethylene (5% w/w);
(e) cetyl ether (5% w/w);
(f) carbopol 940 (1% w/w); and
(g) an aqueous buffer solution adjusted to an appropriate pH (e.g. in the range 6.5-7.5).

Components of the formulation are filter sterilised by passage through a 0.22 mm pore filter as necessary and the formulation is stored at 4 °C until required.

The formulation of the example can be used to treat a subject suffering from hyperproliferative keratinocyte disorders of the skin and/or mucous membrane, such as actinic keratoses or acne.

The skin and/or mucous membrane, as appropriate, is treated with above topical preparation. The cream is topically applied to the entire area to be treated, at ambient temperature, about 18-20 °C. The formulation is left on the subject's skin for 20 minutes and then the area is exposed to daylight for 2 hours. After this the desired the area or washed with soap and water and the patient remains indoors for the rest of the day. The treatment is applied once and the result of treatment is evaluated after 1 month. If desired the treatment can be repeated once after a 3 month interval.

### REFERENCES

1. Monfrecola G, Megna M, Rovati C, Arisi M, Rossi M, Calzavara-Pinton I, Fabbrocini G, Calzavara-Pinton P., A Critical Reappraisal of Off-Label Use of Photodynamic Therapy for the Treatment of Non-Neoplastic Skin Conditions. Dermatology. 2021;237(2):262-276.
2. Assikar S, Labrunie A, Kerob D, Couraud A, Bédane C. Daylight photodynamic therapy with methyl aminolevulinate cream is as effective as conventional photodynamic therapy with blue light in the treatment of actinic keratosis: a controlled randomized intra-individual study. J Eur Acad Dermatol Venereol. 2020 August;34(8):1730-1735
3. Zhang Y, Lin H, Guo X, Zou X. A case series pilot study on the combination of 5-aminolevulinic acid and photodynamic therapy (ALA-PDT) for treatment of vitiligo. An Bras Dermatol. 2018 July-August;93(4):539-545.
4. Wojewoda K, Gillstedt M, Tovi J, Salah L, Wennberg Larkö AM, Sjöholm A, Sandberg C. Optimizing treatment of acne with photodynamic therapy (PDT) to achieve long-term remission and reduce side effects. A prospective randomized controlled trial. J Photochem Photobiol B. 2021 October; 223:112299.
5. Gunaydin G, Gedik ME and Ayan S (2021) Photodynamic Therapy-Current Limitations and Novel Approaches. Front. Chem. 9:691697
6. Lee TH, Kang TH. DNA Oxidation and Excision Repair Pathways. Int J Mol Sci. 2019;20(23):6092. Published 2019 Dec. 3
7. Katsube T, Mori M, Tsuji H, Shiomi T, Wang B, Liu Q, Nenoi M, Onoda M. Most hydrogen peroxide-induced histone H2AX phosphorylation is mediated by ATR and is not dependent on DNA double-strand breaks. J Biochem. 2014 August;156(2):85-95. Epub 2014 Mar. 28.
8. Blackford A N, Jackson SP. ATM, ATR, and DNA-PK: The Trinity at the Heart of the DNA Damage Response. Mol Cell 2017 (66):801-817
9. Ongenae, K., Van Geel, N. and Naeyaert, J.-M. (2003), Evidence for an Autoimmune Pathogenesis of Vitiligo. Pigment Cell Research, 16:90-100
10. Jann N. Sarkaria, Ericka C. Busby, Randal S. Tibbetts, Pia Roos, Yoichi Taya, Larry M. Karnitz and Robert T. Abraham, Inhibition of ATM and ATR Kinase Activities by the Radiosensitizing Agent, Caffeine, Cancer Res 1 1999 (59) (17) 4375-4382
11. Ravi Pate R I, Chiu S M, Machtay M and Oleinick N, Interference with repair of sub-lethal radiation damage by ABT-888, an inhibitor of poly(ADP-ribose) polymerase (PARP), and theobromine [abstract]. In: Proceedings of the 103rd Annual Meeting of the American Association for Cancer Research; 2012 Mar. 31-April 4; Chicago, IL. Philadelphia (PA): AACR; Cancer Res 2012;72(8 Suppl): Abstract nr 5716.
12. Malki A M, Gentry J, Evans S C. Differential effect of selected methylxanthine derivatives on radiosensitization of lung carcinoma cells. Exp Oncol. 2006 March;28(1):16-24.
13. Johnson FE, Harrison B R, McKirgan L W, Raju PI, Roy T K, Virgo KS. A phase II evaluation of pentoxifylline combined with radiation in the treatment of brain metastases. Int J Oncol. 1998 October;13(4):801-5.
14. Conney A H, Lu Y P, Lou Y R, Kawasumi M, Nghiem P. Mechanisms of Caffeine-Induced Inhibition of UVB Carcinogenesis. Front Oncol. 2013; 3:144. Published 2013 Jun. 17.
15. Monteiro J P, Alves M G, Oliveira P F, Silva B M. Structure-Bioactivity Relationships of Methylxanthines: Trying to Make Sense of All the Promises and the Drawbacks. Molecules. 2016 Jul. 27;21(8):974.
16. Ferrucci L M, Cartmel B, Molinaro A M, Leffell DJ, Bale A E, Mayne S T. Tea, coffee, and caffeine and early-onset basal cell carcinoma in a case-control study. Eur J Cancer Prev. 2014;23(4):296-302.
17. Nieder C, Zimmerman FB, Adam M, Molls M. The role of pentoxifylline as a modifier of radiation therapy. Cancer Treatment Reviews, Volume 31, Issue 6, 448-455
18. Dall'Oglio F, Nasca M R, Fiorentini F, Micali G. Diet and acne: review of the evidence from 2009 to 2020, Int J Dermatol. 2021 June;60(6):672-685.
19. Pazzaglia S, Pioli C. Multifaceted Role of PARP-1 in DNA Repair and Inflammation: Pathological and Therapeutic Implications in Cancer and Non-Cancer Diseases. Cells. 2020; 9(1):41.
20. Ha HC and Snyder SH. Poly(ADP-ribose) polymerase is a mediator of necrotic cell death by ATP depletion. PNAS 96(24) 13978-13982
21. Horton JK and Wilson SH (2013) Strategic combination of DNA-damaging agent and PARP inhibitor results in enhanced cytotoxicity. Front. Oncol. 3:257
22. Robu M, Shah RG, Petitclerc N, Brind'Amour J, Kandan-Kulangara F, Shah GM.

Role of poly(ADP-ribose) polymerase-1 in the removal of UV-induced DNA lesions by nucleotide excision repair. Proc Natl Acad Sci USA. 2013 Jan. 29;110(5):1658-63.
23. C. Anne-Marie Couto, Hong-Yu Wang, Joanna C.A. Green, Rhian Kiely, Robert Siddaway, Christine Borer, Catherine J. Pears, Nicholas D. Lakin; PARP regulates nonhomologous end joining through retention of Ku at double-strand breaks. J Cell Biol 8 Aug. 2011; 194 (3): 367-375
24. Helleday T, Bryant HE, Schultz N. Poly(ADP-ribose) polymerase (PARP-1) in homologous recombination and as a target for cancer therapy. Cell Cycle. 2005 September;4(9):1176-8.
25. Liesbeth Geraets, Harald J.J. Moonen, Emiel F.M. Wouters, Aalt Bast, Geja J. Hageman, Caffeine metabolites are inhibitors of the nuclear enzyme poly(ADP-ribose)polymerase-1 at physiological concentrations, Biochem. Pharm. 2006, 72(7), 902-910

1. A first composition comprising photodynamic therapy photosensitizer, and a second composition comprising a triple (Ataxia Telangiectasia Mutated (ATM) kinase, Ataxia Telangiectasia Related (ATR) kinase, Poly-ADP ribose polymerase (PARP)) inhibitor, preferably theobromine, for the use in the treatment of a mammalian, preferably human, subject.

2. A first composition comprising photodynamic therapy photosensitizer, and a second composition comprising a triple (ATM, ATR, PARP) inhibitor according to claim 1, preferably theobromine, for the use in the treatment of a disorder of the skin and/or mucous membrane of a mammalian, preferably human, subject.

3. A first composition comprising photodynamic therapy photosensitizer, and a second composition comprising a triple (ATM, ATR, PARP) inhibitor according to claim 1, preferably theobromine, for the use in the treatment of a hyperproliferative keratinocyte disorder of the skin and/or mucous membrane of a mammalian, preferably human, subject.

4. A first composition comprising photodynamic therapy photosensitizer, and a second composition comprising a triple (ATM, ATR, PARP) inhibitor according to claim 1, preferably theobromine, for the use in the treatment of actinic keratosis of a mammalian, preferably human, subject.

5. A first composition comprising photodynamic therapy photosensitizer, and a second composition comprising a triple (ATM, ATR, PARP) inhibitor according to claim 1, preferably theobromine, for the use in the treatment of acne of a mammalian, preferably human, subject.

6. A first composition comprising photodynamic therapy photosensitizer, and a second composition comprising a triple (ATM, ATR, PARP) inhibitor according to claim 1, preferably theobromine, for the use in the treatment of a disease of a mammalian, preferably human, subject.

7. A first composition comprising photodynamic therapy photosensitizer, and a second composition comprising a triple (ATM, ATR, PARP) inhibitor according to claim 1, preferably theobromine, for the use in the treatment of basal cell carcinoma of a mammalian, preferably human, subject.

8. A composition comprising photodynamic therapy photosensitizer and a triple enzyme inhibitor (Ataxia Telangiectasia Mutated kinase, Ataxia Telangiectasia Related kinase and poly-(ADP-ribose) polymerase inhibitor).

9. A composition according to claim 1, formulated for topical application to the skin and/or mucous membrane of a mammalian, preferably human, subject.

10. A composition according to claim 1, formulated for oral administration to a mammalian, preferably human, subject.

11. (canceled)

12. A composition according to claim 1, wherein the composition comprises a broad inhibitor of Ataxia Telangiectasia Mutated kinase, Ataxia Telangiectasia Related kinase and poly(ADP-ribose) polymerase which is theobromine or any other member of the methylxanthine family of compounds that has the triple inhibitory activity, preferably theobromine.

13. A composition according to claim 1, wherein the composition comprises a photodynamic therapy photosensitizer, selected from the group consisting of: methyl aminolevulinate or 5-aminolaevulinic acid hematoporphyrin derivative, m-tetrahydroxophenyl chlorine, mono-L-aspartyl chlorine e6, fotosens, phthalocyanines, benzoporphyrins, chlorins, bacteriochlorins, naphthalocyanines; and further wherein the photodynamic therapy photosensitizer is preferably 5-aminolaevulinic acid and even more preferably methyl.

14. A composition according to claim 1 further comprising one or more of the substances selected from the group consisting of:
emulsifiers; preservatives and antimicrobials; solvents, carriers, or diluents; and thickeners and emollients.

15. A composition according to claim 11, comprising one or more of: water;
parabens; polysorbates; buffers; and glycerol.

16. (canceled)

17. (canceled)

18. (canceled)

19. A composition according to claim 1, comprising one or more substances selected from the group consisting of: diclofenac; imiquimod; 5-fluorouracil; salicylic acid; ingenol mebutate; tirbanibulin; benzoyl peroxide; adapalene; tretinoin; isotretinoin; antibiotics including clindamycin, erythromycin, lymecycline; dapsone.

20. A composition according to claim 1, comprising photodynamic therapy photosensitizer at a concentration in the range 0.01 to 50% w/w or w/v, preferably in the range 0.1 to 30%, and more preferably in the range 1 to 20% and most preferably in the range 7-16%.

21. A composition according to claim 1, comprising a triple (ATM, ATR, PARP) inhibitor, preferably theobromine a concentration in the range 0.001 to 50% w/w or w/v, preferably in the range 0.01 to 10%, and more preferably in the range 0.05 to 1%, and most preferably in the range 0.1-0.5%, the compound being preferably theobromine.

22. A method of making a composition in accordance with claim 8, the method comprising the step of: causing to be present, in a single composition, an effective concentration of photodynamic therapy photosensitizer and an effective concentration of the triple (ATM, ATR, PARP) inhibitor, preferably theobromine.

23. A method according to claim 22, further comprising the step of mixing photodynamic therapy photosensitizer and the triple (ATM, ATR, PARP) inhibitor with an acceptable carrier, diluent, buffer, antimicrobial or excipient, and packaging the composition in a container.

24. A method according to claim 22, wherein the triple (ATM, ATR, PARP) inhibitor is a methylxanthine, preferably theobromine.

25. (canceled)

26. (canceled)
